(19) United States
(12) Patent Application Publication
(10) Pub. No.: US 2007/0225491 A1
(43) Pub. Date: Sep. 27, 2007

(54) Phthalocyanine Derivatives, Their use as Homeotropically Aligned Layer in Electronic Devices and Method for the Manufacturing Thereof

(76) Inventors: Vinciane De Cupere, Court-Saint-Etienne (BE); Tant Julien, Brussels (BE); Geerts Yves, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/588,865

(22) PCT Filed: Jan. 18, 2005
(86) PCT No.: PCT/EP05/00556

§ 371 Date: Jun. 8, 2007

(30) Foreign Application Priority Data
Feb. 10, 2004 (EP) 04447032.6

(51) Int. Cl. H01L 51/30; C07D 487/22; C07F 1/08; C07F 15/00; C07F 15/04; C07F 3/06; C09K 19/34

(52) U.S. Cl. 540/141; 540/139

(57) ABSTRACT

The present invention discloses a liquid crystalline tetra alkyloxy-substituted phthalocyanine derivative with the following structure: (I) 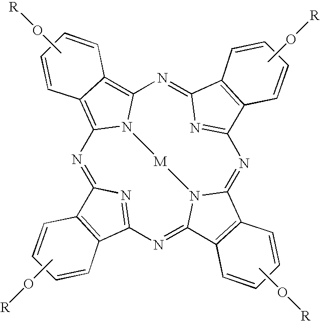
where M is a metal such as zinc (Zn), copper (Cu), platinum (Pt), palladium (Pd), or two atoms such as 2 H or 2 Li, and R is the followed branched aliphatic chain: (II) 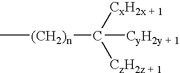 with n=0 and x=6-30 y=6-30 z=0-30 or n=1 and x=10-30 y=6-30 z=0-30 or n>1 and x=6-30 y=6-30 z=6-30.

### FIELD OF THE INVENTION

The present invention relates to new liquid crystalline phthalocyanine derivatives, to a method for preparing the same and to their use in electronic devices.

### STATE OF THE ART

Discotic liquid crystals have been extensively described by Oswald and Pieranski (Les Cristaux Liquides, tome 1 and 2, Gordon and Breach Science Publishers, Paris). They usually consist in a rigid aromatic core surrounded by several flexible side chains. Those materials are known for their ability to self-organise in columns, forming a quasi-one dimensional semi-conductor (Boden N., Bushby R. S., Clement S., J. Chem. Phys., 1993, 98(7), 5920). Indeed, the stacking of the aromatic cores leads to the formation of conductive wires while the side chains act as an insulating coating, allowing the charges and the excitons to move only in the direction perpendicular to the plane of the conjugated cores.

It has been shown that, due to this anisotropy, the long range conductivity of such materials strongly depends on the molecular organisation in electronic devices. The best configuration is obtained when the columns, and by the way, the optical director of the material, are perpendicular to the electrodes (FIGS. 1a and 1b). Organisation presented in 1b is observed for a material presenting a columnar rectangular phase in which the disks are tilted in the columns. The preferred molecular organisation is then the homogeneous alignment in the case of Field Effect Transistors (FET) and the homeotropic alignment in all the other devices (Organic Light Emitting Devices (OLED), Photovoltaic Cells (PVC), sensors). Another suitable configuration is obtained when the optical director of the material forms a 70° to 90° angle with respect to the electrode surface, while the disks are still parallel to said surface (FIG. 1c). As the organisation depicted in FIG. 1b, this last case is observed for a material presenting a columnar rectangular phase. Such considerations have been approached in documents WO 9636082, EP1028475, EP 1450420 and WO 03023506. The latter two consider more specifically the FET configuration in which homogeneous alignment is used. The others mention the obtaining of homeotropic alignment, by use of an alignment layer (EP 1028475) or without giving any information about the procedure to apply to obtain the expected organisation (WO 9636082).

Hatsusaka et al. (J. Mater. Chem. (2001), 11, 423) have showed that large homeotropically aligned domains can be obtained by slow cooling of phthalocyanine derivatives from the isotropic phase to the columnar tetragonal phase. However, the temperature range in which said alignment is obtained is very narrow (between 149.5 and 187.5° C.) and observed only between two soda lime glass and quartz glass plates.

Here we disclose a method for preparing materials having a clearing point below their decomposition temperature and forming spontaneously homeotropic alignment between two surfaces, on a wide range of temperatures including ambient, and on a large variety of substrates.

### AIMS OF THE INVENTION

The present invention aims to provide new phthalocyanine derivatives and a preparation method thereof.

In particular, the present invention provides tetra alkyloxy-substituted phthalocyanine derivatives with specific functionalization, optimised to obtain low clearing point and homeotropic alignment when sandwiched between two plates.

### SUMMARY OF THE INVENTION

The present invention discloses a liquid crystalline tetra alkyloxy-substituted phthalocyanine derivative with the following structure I: 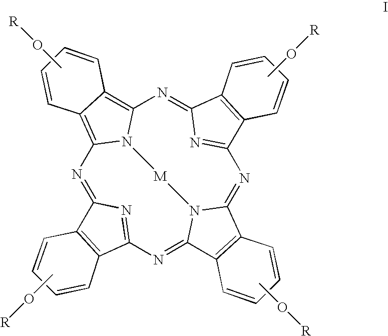
wherein M is a metal or two atoms such as 2 H or 2 Li, and R is the followed branched aliphatic chain: 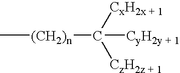
with n=0 and
x 6-30
y 6-30
z=0-30
   or n=1 and
x=10-30
y=6-30
z=0-30
   or n>1 and
x=6-30
y=6-30
z=6-30.

According to a particular embodiment, the invention comprises one or several of the following features:
M is 2H or 2 Li and n=1, x=12, y=10 and z=0;
M is 2H or 2 Li and n=1, x=10, y=8 and z=0;
M is Copper (Cu), Zinc (Zn) Palladium (Pd), Ni (Nickel) or Pt (platinum) and n=1, x=12, y=10 and z=0;
M is Copper (Cu), Zinc (Zn) Palladium (Pd), Ni (Nickel) or Pt (platinum) and n=1, x=10, y=8 and z=0;

Moreover, the present invention also discloses a preparation process of the phthalocyanine derivatives, comprising the following steps:
reacting nitrophthalonitrile II in dimethyl sulfoxide (DMSO) with at least the molar amount of an inorganic base (lithium hydroxide (LiOH), potassium hydroxide (KOH), sodium hydroxide (NaOH), . . . ), and with at least the molar amount of an alcohol III, by reacting the mix up at 0-60° C. during at least 10 hours;
separating the alkoxyphthalonitrile IV from the resulting reaction medium comprising said compound, remaining solvents, unused reactants and by-products;
reacting the alkoxyphthalonitrile IV in 1-pentanol or N,N-dimethylethanolamine with at least 2 times the molar amount of lithium (Li), by reacting the mix up at reflux during at least 2 hours;
if the non-metal phthalocyanine (M=2H) is needed, acetic acid is added to the reaction medium; if a metal phthalocyanine is needed, at least one time the theoretical amount of the corresponding metal salt (acetate, chloride, bromide, . . . ) is added to the reaction medium and left at reflux for at least 30 minutes; and
separating the tetrasubstituted phthalocyanine I from the resulting reaction medium comprising said compound, remaining solvents, unused reactants and by-products.

The present invention also discloses the use of the tetra alkyloxy-substituted phthalocyanines in electronic devices.

Finally, the present invention further discloses the use of the tetra alkyloxy-substituted phthalocyanines in electronic devices such as field effect transistors, sensors, memories, photovoltaic devices and photodiodes.

### SHORT DESCRIPTION OF THE DRAWINGS

FIG. 1 represents the molecular organisations which can be obtained when a suitable discotic liquid crystal is sandwiched between two plates: a) and b) homeotropic alignment, c) alignment for which the optical director forms an angle lower than 90° with respect to the surface. The director (N) is represented by an arrow.

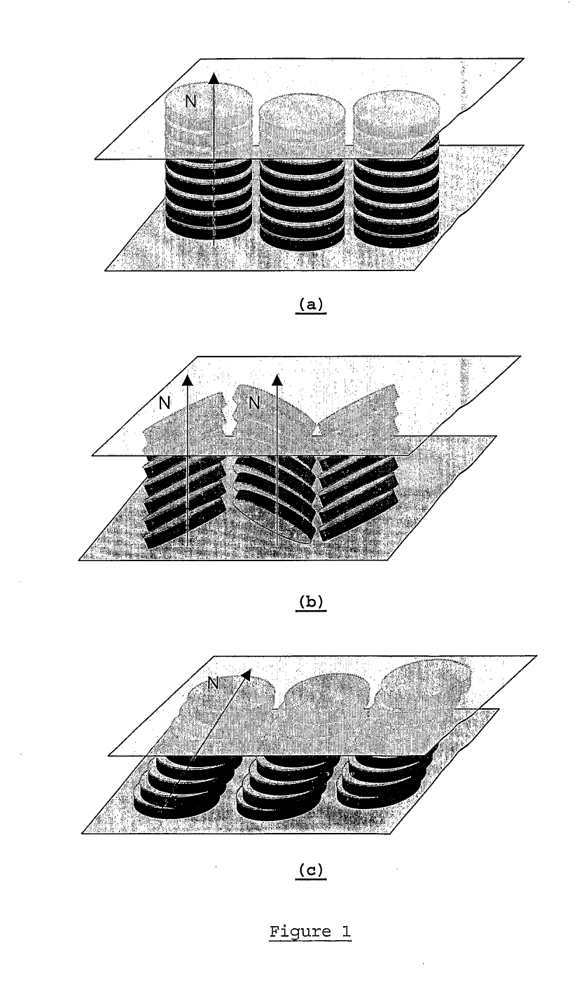

FIG. 2 represents the synthetic scheme used to obtain the phthalocyanine derivatives.

FIG. 3 represents schematically an electronic device comprising an homeotropically aligned layer of phthalocyanine derivative I (layer 1) sandwiched between to substrates (layer 2) and (>layer 3), said substrates being constituted, independently, of a glass related or polymer layer (a), possibly coated with a metal or a metal oxide layer (b) and with a light emitting or semi-conducting material (c).

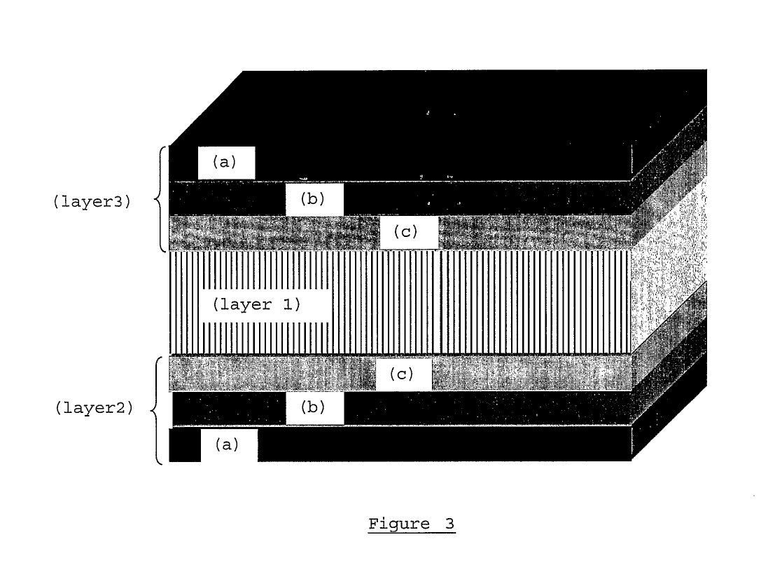

### DETAILED DESCRIPTION OF THE INVENTION

The present invention concerns tetra alkyloxy-substituted phthalocyanine derivatives and their preparation process.

The present invention discloses phthalocyanine derivatives of the structure I 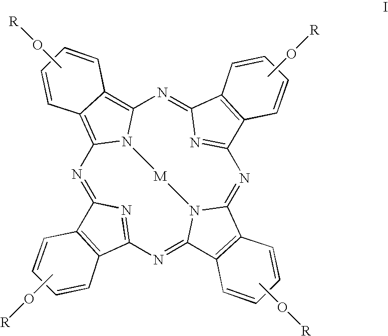
wherein M is, without being limitative, a metal such as zinc (Zn), copper (Cu), platinum (Pt), palladium (Pd), . . . or two atoms such as 2 H or 2 Li, and R is the following branched aliphatic chain: 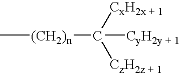
with n=0 and
x=6-30
y=6-30
z=0-30
   or n=1 and
x=10-30
y=6-30
z=0-30
   or n>1 and
x=6-30
y=6-30
z=6-30

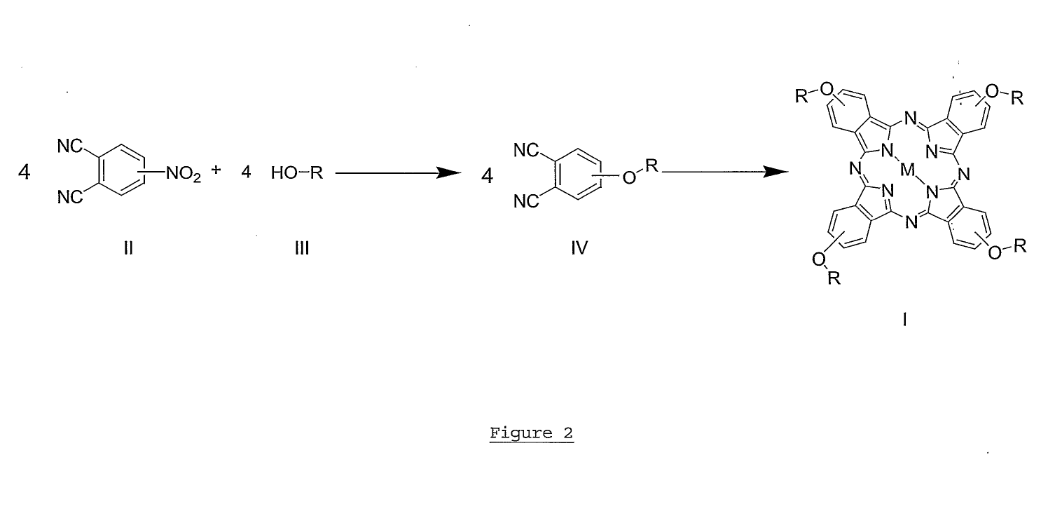

The preparation of tetrasubstituted phthalocyanine derivatives I comprises the following steps (FIG. 2):
a) Reacting nitrophthalonitrile II in dimethyl sulfoxide (DMSO) with at least the molar amount of an inorganic base (lithium hydroxide (LiOH), potassium hydroxide (KOH), sodium hydroxide (NaOH), . . . ), and with at least the molar amount of an alcohol III, by reacting the mix up at 0-60° C. during at least 10 hours;
b) separating the alkoxyphthalonitrile IV from the resulting reaction medium comprising said compound, remaining solvents, unused reactants and by-products;
c) reacting the alkoxyphthalonitrile IV in 1-pentanol or N,N-dimethylethanolamine with at least 2 times the molar amount of lithium (Li), by reacting the mix up at reflux during at least 2 hours;
d) if the non-metal phthalocyanine (M=2 H) is needed acetic acid is added to the reaction medium; if a metal phthalocyanine is needed, at least one time the stoechiometric amount of the corresponding metal salt (acetate, chloride, bromide, . . . ) is added to the reaction medium and left at reflux for at least 30 minutes; and
   e) separating the tetrasubstituted phthalocyanine I from the resulting reaction medium comprising said compound, remaining solvents, unused reactants and by-products.

Preferably, the process employs dry reaction conditions (solvents, glassware, . . . ). Preferably also, the process is done under inert atmosphere (nitrogen or argon).

The molecules, soluble in common organic solvents, are characterised by ¹H NMR, mass spectroscopy and absorption spectroscopy. Their thermotropic behaviour is characterised by cross-polarised microscopy, DSC and X-ray diffraction.

The obtained compounds present clearing points below their decomposition temperature and spontaneously form homeotropic alignment when sandwiched between two plates, over a wide range of substrates and temperatures including usual working temperatures for electronic devices.

The obtained compounds can be used to build electronic devices comprising an homeotropically aligned layer of them. Said method comprises the following steps:
a) Depositing a 50 nm to 15 μm thick layer of one of the phthalocyanine derivatives I (layer 1) on a first substrate (layer 2) and covering said phthalocyanine derivative layer (layer 1) by the second substrate (layer 3) to build a sandwiched device. Layers 2 an 3 can be identical or different, depending on the application, and will be described in details later in the text.
   The phthalocyanine derivative film (layer 1) can be deposited on the first substrate (layer 2) by spin-coating, doctor blading, zone casting, or any other suitable technique.
b) Heating the obtained sandwiched device at a temperature slightly above the isotropic transition temperature of the phthalocyanine derivative I. Applying a slight pressure on the upper substrate (layer 3) in order to ensure an intimate contact with the phthalocyanine film (layer 1).
c) Cooling down the sandwiched device at a cooling rate lower or equal to 20°/min to a temperature well below the isotropisation temperature.

The substrates (layers 2 and 3) can be, independently, soda lime glass, silicon or quartz (a) coated by metal or metal oxide (b) in order to provide electrodes. Typical coating materials are the following: silver, gold, aluminium, magnesium, calcium, indium tin oxide, tin oxide, zinc oxide, titanium oxide, gallium oxide, yttrium oxide, praseodymium oxide or any other suitable metal or metal oxide.

Advantageously, the substrates (layers 2 and 3) can be, independently, polymer plates (a) coated with metal or metal oxide (b). Without being limitative, good candidates for such substrates are the following: polytetrafluoroethylene, polyethylene-(terephthalate), polycarbonate, polyvinylchloride, poly-urethane, polypropylene, poly(methyl methacrylate).

Substrates (layers 2 and 3) can also be independently constituted of glass or polymer plates (a) coated with metal or metal oxide (b) and covered with semi-conducting or light emitting polymers (c). Semi-conducting polymers can be used to make the injection of charges in the system easier and/or to smooth the surface of the electrodes. Such polymer can also be used to build PVCs, where two distinct semi-conducting materials are needed, an electrons carrier (n-type material) and an hole carrier (p-type material). In the present invention, the phthalocyanine derivative can be used as an hole or an electron carrier, depending on the material with which it is combined. Semi-conducting polymers can be, without being limitative, PEDOT-PSS, polyoxadiazoles, poly(9,9-dioctylfluorene-co-benzothiadiazole), poly(9,9-dioctyl-fluorene), poly-pyridines, polyquinoxalines, poly-quinolines, . . . Light emitting properties are useful for the design of OLEDs, where photo-emissive active layer is needed. Light emitting polymers can be, without being limitative, poly(pyridine) derivatives, poly(p-phenylene-vinylene) derivatives, polyfluorene derivatives, poly-(acetylene) derivatives, poly(thiophene) derivatives, . . . Such polymers can be deposited by spin-coating, doctor-blading, solvent casting, zone casting, . . .

Advantageously, substrates (layers 2 and 3) can also be constituted of glass or polymer plates (a) coated with metal or metal oxide (b) and covered with liquid crystalline, crystalline or amorphous semi-conducting or light emitting molecular materials (c), used in the same way as semi-conducting or light emitting polymers. Examples of such molecular materials are: hexaazatriphenylenes, hexaazatrinaphthylenes, dodecaazatrinaphthylenes, hexa-azatri-isooxanaphthylenes, hexa-azatriisothianaphthylenes, tricycloquinazolines, perylo[1,12-efg]isoindole-1,3-dione, tetraaza-tetrahydrocoronene-tetracarboxylic acid bisphenyl-imide, terylenes, quaterylenes, perylenes, pyrenes, perinones bisbenzimidazole, pentacenes, anthracenes, rhodamine and fullerenes, . . . especially C61-butyric acid methyl ester. Such molecules can be deposited by spin-coating, solvent casting, zone casting, doctor-blading, vapour deposition or any other suitable technique.

### EXAMPLE 1

### Synthesis of 2(3),9(10),16(17),23(24)-Tetra(2-decyltetradecyloxy)-phthalocyanine

n=1, x=12, y=10 and z=0

A mixture of 4-nitrophthalonitrile II (25 mmol) and the 2-tetradecanol III (40 mmol) in 100 mL of anhydrous methylsulfoxide is stirred during two hours at RT. Lithium hydroxide powder (50 mmol) is then added with stirring. The reaction medium turns from yellow to black, and is stirred 3 days at RT. The solution is poured in water and is extracted three times with ethyl acetate. The combined organic fractions are dried on Na₂SO₄, filtrated, and evaporated. The crude products (a dark green-yellow oil) is purified on a silica gel column chromatography with toluene as eluent to afford the pure 4-(2-tetradecyloxy)-phthalonitrile IV as a viscous light yellow oil, with yields of ranging from 50-57 W.

The 4-(2-tetradecyloxy)-phthalonitrile IV (2 mmol) is mixed with a large excess of metal lithium, in 6 mL of dry 1-pentanol. The reaction mixture is then heated to reflux under inert atmosphere. After 4 hours, 30 mL of acetic acid is added to the dark green solution. The formed precipitate is collected by filtration, and washed with water and methanol. The pasty green material obtained is then dissolved in methylene chloride, and the solvent is evaporated under vacuum. The pure product is obtained after purification on silica gel column chromatography (toluene/hexane 1:1 as eluent) to afford I in yields ranging from 43-50%.

### EXAMPLE 2

### Manufacturing of a Photovoltaic Cell

An electronic device comprising 2(3),9(10),16(17),23(24)-tetra(2-decyltetradecyloxy)-phthalocyanine I (layer 1) with lateral chain with n=1, x=12, y=10 and z=0, homeotropically aligned, sandwiched between a first substrate (layer 2) constituted by a glass substrate (a) coated with Indium Tin Oxyde (ITO) (b) and a second substrate (layer 3) constituted by a glass plate (a) coated with Aluminium (Al) (b) and spin coated with a C61-butyric acid methyl ester (PCBM) layer (c).

The device is obtained with the following manufacturing method:
A glass plate (a) covered by an Al electrode (b) is spin-coated with a PCBM solution (4 g/l in toluene) at 1500 rpm with a rate of 1500 rpm/sec in order to obtain a first substrate (layer 3).
A 100 to 300 nm thick layer of the phthalocyanine derivative I (layer 1) is deposited on a second substrate consisting in an ITO coated glass plate (layer 2) and covered with the Al/PCBM coated glass plate (layer 3).
The obtained sandwiched device is heated on a hot plate at 200° C., in order to reach the isotropic (liquid) phase of the phthalocyanine derivative. A slight pressure is applied on the second substrate (layer 3) in order to ensure an intimate contact between layer 1 and layer 3.
The sandwiched device is cooled at a rate of 10° C./min down to ambient temperature.

1. A liquid crystalline tetra alkyloxy-substituted phthalocyanine derivative with the following structure I: 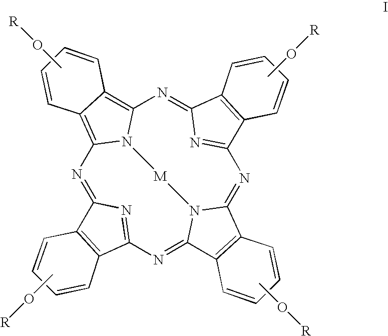
wherein M is a metal or two atoms such as 2 H or 2 Li, and R is the followed branched aliphatic chain: 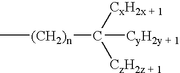
with n=0 and
x=6-30
y=6-30
z=0-30
or n=1 and
x=10-30
y=6-30
z=0-30
or n>1 and
x=6-30
y=6-30
z=6-30.

2. The phthalocyanine derivative according to claim 1, where M is 2 H or 2 Li and n=1, x=12, y=10 and z=0.

3. The phthalocyanine derivative according to claim 1, where M is 2 H or 2 Li and n=1, x=10, y=8 and z=0.

4. The phthalocyanine derivative according to claim 1, where M is Copper (Cu), Zinc (Zn), Palladium (Pd), Ni (Nickel), or Pt (platinum) and n=1, x=12, y=10 and z=0.

5. The phthalocyanine derivative according to claim 1, where M is Copper (Cu), Zinc (Zn), Palladium (Pd), Ni (Nickel), or Pt (platinum) and n=1, x=10, y=8 and z=0.

6. Preparation process of the phthalocyanine derivative according to claim 1, comprising the following steps:
reacting nitrophthalonitrile II in dimethyl sulfoxide (DMSO) with at least the molar amount of an inorganic base, and with at least the molar amount of an alcohol III, by reacting the mix up at 0-60° C. during at least 10 hours;
separating the alkoxyphthalonitrile IV from the resulting reaction medium comprising said compound, remaining solvents, unused reactants and by-products;
reacting the alkoxyphthalonitrile IV in 1-pentanol or N,N-dimethylethanolamine with at least 2 times the molar amount of lithium (Li), by reacting the mix up at reflux during at least 2 hours;
if the non-metal phthalocyanine (M=2 H) is needed acetic acid is added to the reaction medium; if a metal phthalocyanine is needed, at least one time the theoretical amount of the corresponding metal salt is added to the reaction medium and left at reflux for at least 30 minutes; and
separating the tetrasubstituted phthalocyanine I from the resulting reaction medium comprising said compound, remaining solvents, unused reactants and by-products.

7. (canceled)

8. (canceled)

9. The process of claim 6 wherein the inorganic base is selected from the group consisting of lithium hydroxide, potassium hydroxide and sodium hydroxide.

10. An electronic device comprising the phthalocyanine derivative of claim 1.

11. The electronic device of claim 10 which is selected from the group consisting of transistors, sensors, memories, photo voltaic devices and photodiodes.
